(19) United States
(12) Patent Application Publication
(10) Pub. No.: US 2024/0307625 A1
(43) Pub. Date: Sep. 19, 2024

(54) PROTECTIVE SHEATH FOR A GLASS PHARMACUETICAL CARTRIDGE, SYSTEM, AND METHOD OF MANUFACTURE

(71) Applicant: Rx Bandz, Inc., Locust Valley, NY (US)

(72) Inventor: Stephen HARHEN, Rumson, NJ (US)

(21) Appl. No.: 18/185,121

(22) Filed: Mar. 16, 2023

Related U.S. Application Data
(63) Continuation of application No. PCT/US2021/051063, filed on Sep. 20, 2021.
(60) Provisional application No. 63/080,258, filed on Sep. 18, 2020.

(51) Int. Cl. A61M 5/28

(52) U.S. Cl. CPC A61M 5/282; A61M 2207/00

(57) ABSTRACT

The sheathed pharmaceutical cartridges described herein are resistant to structural damage such as burst and damage that can occur in and during activation of an auto-injector system. The cartridges having the protective sheath described herein are resistant to damaging forces generated in austere or demanding environments. In one embodiment, the glass cartridge can include a force absorption sheath to distribute and dissipate stress that extends from the outer surface of the body of the glass and positioned around at least a portion of the outer surface. In one embodiment, the recovered inner diameter of the sheath post-manufacture applies an inward compressive force on the body of glass.

### CROSS REFERENCE TO RELATED APPLICATION

This application is a continuation of International Patent Application No. PCT/US2021/051063, filed on Sep. 20, 2021, which claims priority to and claims the benefit of U.S. Provisional Application No. 63/080,258, filed on Sep. 18, 2020. The disclosures of each of which are herein incorporated by reference in their entirety.

### FIELD

The field of the disclosed subject matter is related to a protective sheath for a pharmaceutical glass cartridge for medical devices, such as for purposes of example and not limitation, shatter and burst resistant, ruggedized, pharmaceutical glass cartridges for auto-injectors.

### BACKGROUND

Glass cartridges have found widespread use for the storage and delivery of pharmaceutical medicines and therapeutics, in both civilian and military applications. For example, Type I glass cartridges are sterile, low cost, can hold a variety of medications and therapeutics, have zero leaching, and allow for easy viewing of their contents. Additionally, they can accommodate and be suitable for containing a variety of pharmaceutical formulations. Such cartridges can be employed in a wide range of injectors, including auto-injectors.

Historically, glass has been used for packaging pharmaceuticals because of its hermeticity, optical clarity, and chemical durability relative to certain other materials. Specifically, the glass used in pharmaceutical packaging can have adequate chemical durability and must not affect the stability of the pharmaceutical formulations contained therein. Glasses having suitable chemical durability include those glass compositions within the United States Pharmacopoeia (USP)<660>Type I, II, or III glass compositions.

Although these glass compositions are commonly used in pharmaceutical cartridges, they can suffer from several deficiencies. The use of glass in pharmaceutical packaging can be limited by the mechanical performance of the glass. Specifically, the high speeds and impact forces experienced during auto-injector activation can result in mechanical damage on the surface of the glass, such as fractures and the like, as the cartridge can come into contact with a needle-hub, interior components, and/or interior walls of the auto-injector. This mechanical damage can decrease the strength of the glass pharmaceutical cartridge resulting in an increased likelihood that cracks will develop in the glass, potentially compromising the ability of the pharmaceutical agent contained in the cartridge to be delivered to the subject in need. Conventional glass pharmaceutical cartridges can have bonded (adhesives or chemical) coatings and tapes to assist with strengthening the cartridges. Unfortunately, these coatings and sheaths are not necessarily sufficient to absorb significant blunt forces or protect or contain the cracks that can develop during manufacture and during the field of use.

Glass cartridges have many advantages, but they are further susceptible to damage that can occur in demanding or austere environments. In military use, they can be employed on or near the battlefield. While robust, they are not necessarily able to withstand certain rigors of military use, including shocks and impacts experienced when carried by soldiers. In both civilian and military use, the cartridges can be susceptible to damage and debris can penetrate the auto-injector.

It can be desirable for such auto-injectors to be ruggedized, but such is not always feasible due to the substantially greater cost of a ruggedized auto-injector as compared with a standard consumer-grade auto-injector. Indeed, a ruggedized auto-injector can cost many times as much as a regular, consumer-grade version. “Ruggedized” auto-injectors as used herein is a broad term, and is to be given its ordinary and customary meaning to a person of ordinary skill in the art (and is not to be limited to a special or customized meaning), and refers without limitation to an auto-injector device that resists damage due to harsh environmental conditions or abuse, such as impact, vibration and shock, dust and dirt, moisture and liquid spills, and extreme heat and cold. The harsh environment of a police or other emergency vehicles, job sites, recreational and military environments can subject an ordinary auto-injector to some or all the above-listed conditions, causing the internal cartridge to fail. The need to create a ruggedized cartridge independent of the auto-injector serves to provide direct protection from forces occurring in the external environment and during auto-injector activation.

Upon activation, the cartridge or cartridge component advances forward towards the needle-hub. The cartridge is urged to advance via release of stored energy. This rapid advancement puts increased stresses and pressures on the cartridge increasing the risk of generating or propagating a crack from a critical defect in the glass. Forces of concern include but are not limited to the impact force on the shoulder of the cartridge as it is inserted into the needle-hub and increased internal pressure generated during drug or therapeutic extrusion.

Likewise, the glass cartridge manufacturing process can also cause damage, including hairline and other cracks in the glass cylinder. Certain defects can be hard to identify as critical in the quality management system.

Accordingly, a need exists for improving glass containers for use as pharmaceutical and therapeutic cartridges which have increased strength, and/or damage tolerance to the forces experienced in and during activation of the auto-injector. Furthermore, there is a need for a glass cartridge that utilize a sheath that can protect the cartridge in both civilian and military applications and detect defects occurring from the manufacturing process.

### SUMMARY

The disclosed subject matter provides an unbonded protective sheath for a pharmaceutical or therapeutic glass cartridge that addresses the flaws inherent in certain cartridges, as shown and described herein.

The protective sheath according to the disclosed subject matter includes physical and chemical properties to enable the sheath to shrink over the cartridge post-manufacture and then compress the cartridge and protect the glass cartridge during activation and use. The shrinkage and compression of the protective sheath can detect existing defects by crushing cartridges weakened by a defect and indirectly affix the sheath to the glass without a permanent bond. This is in direct contrast to certain techniques known in the art, such as polymer coatings and lubricious bonded coatings and layers. Such conventional techniques can attempt to prevent defects during high-speed manufacturing processing as opposed to detecting defects during downstream field of use of the cartridge as provided in the disclosed subject matter.

The protective sheath according to the disclosed subject matter is not permanently bonded to the glass cartridge. Rather, the sheath slips over the cartridge and is recovered or shrunk during manufacture to affix itself to the cartridge in a rigid format that is configured to permanently deform. The sheath is unbonded and not permanently affixed to the glass cartridge.

As discovered by the inventor, there are multiple benefits to being not directly bonded permanently to the glass as disclosed herein. The sheath according to the disclosed subject matter provides direct mechanical evidence and inspection of every cartridge through the force applied by the recovered compression of the sheath, as opposed to sputtered or other coatings that are conventionally applied. The sheath according to the disclosed subject matter provides a force absorbing barrier that is not at risk of chemically contaminating the inside of the cartridge. Once applied, the sheath according to the disclosed subject matter remains a rigid, unbonded unit that permits micromovements between the glass and the sheath. Further, the sheath according to the disclosed subject matter provides an ideal surface to identify product abuse (such as impacts thereto), and allows for maintaining the glass cartridge shape and reduces glass contamination in the event of breakage. In one embodiment, the sheath applies a compressive pressure on the glass less than or equal to 150 psi, and more particularly between approximately 75 psi and approximately 100 psi.

A limitation of currently available conventional cartridges is that they are optically inspected with low-latent energy such that minor defects are difficult and challenging to detect. With conventional techniques, mechanical inspection is very costly or not possible on currently available cartridges.

According to the disclosed subject matter, the sheaths disposed on the cartridges provide protection for the cartridge during auto-injector activation and post-manufacture. Other conventional approaches seek to prevent defects in manufacturing processes and assume that the cartridge will survive activation forces, which is an unfortunate assumption. Often auto-injectors, such as for gas and chemical decomposition application, and cartridges in more demanding environments are experiencing failure.

Embodiments of the disclosed subject matter provide significant benefits by the compressive nature of the disclosed sheath, amongst other characteristics as described herein.

According to one embodiment, a protective sheath is disposed on a glass container including a body having an inner surface, an outer surface and a wall thickness extending between the outer surface and the inner surface. For purposes of example and not for limitation, the glass cartridge body can have a suitable thickness dimension such as 1 mm for a 0.5 ml glass cartridge. For purposes of example and not for limitation, the sheath can have a suitable thickness dimension as a function of the wall thickness of the glass container. In one embodiment, the sheath has a thickness dimension less than or equal to 0.2 mm, In certain embodiments, the sheath can have a thickness dimension of less than or equal to 1 mm, For purposes of example, at least the inner surface of the glass cartridge body can have a delamination factor less than or equal to 10. The outer surface can have the disclosed sheath applied thereto to absorb impact forces.

In another embodiment, a glass container can include a body having an inner surface, an outer surface, and a wall thickness extending between the outer surface and the inner surface. For purposes of example and not limitation, the body can be formed from a Type I, Class B glass according to ASTM Standard E438-92. The compressive sheath on the outer surface of the glass cartridge can be positioned to shield some of or all of the glass body.

The protective sheath is furthermore a force absorption sheath. The sheath protecting the glass can be positioned on the surface such that the sheath does not contact the inner surface of the glass cartridge. As such, the sheath can cooperate with the outer surface of the glass cartridge. The sheath can have a minimum diameter post-manufacture and a maximum diameter pre-manufacture. As such, the sheath can have an original inner diameter prior to coupling the sheath to the cartridge such that it can be sleeved over the glass cartridge and a recovered diameter after coupling the sheath (such as by cold or heat shrinking the sheath to the cartridge) with the cartridge to secure itself on the cartridge and apply a compressive force thereto. Once the sheath is coupled with the cartridge and the heat shrink sheath cools post-manufacturing, the diameter of the sheath (or recovered diameter) does not change as the sheath is cured into a rigid unit about the cartridge. However, the sheath can permanently deform at select locations of undue stress imparted upon the inner surface of the sheath, such as at a crack location of the glass cartridge as described herein. In other embodiments, the sheath can be cold shrunk to the glass cartridge during manufacture. In one embodiment the sheath can impart a compressive force on the cartridge up to 150 psi. The sheath can have a minimum diameter greater than or equal to the maximum diameter of a cartridge. Furthermore, the sheath can have a heat shrink ratio ranging typically from 1.6:1 to 4:1

The force absorption sheath can be unbonded or not chemically adhered to the glass cartridge. The force absorption sheath can ‘float’ unbonded from the glass of the cartridge to provide superior protection and ruggedization. As such, the sheath can move in micromovements with respect to the glass cartridge.

The force absorption sheath can be under compression to secure the sheathing around some or all the glass cartridge.

The compression generated by the recovered or reduced inner dimension of the absorption sheath can identify critical defects in the glass and can propagate cracks that would otherwise be undetectable to the eye thereby permitting easier and faster detection of cracking in the vial and aiding quality control.

The force absorption sheath can be positioned on the surface of the glass body such that the heel and shoulder of the glass cartridge is protected from impact forces generated by the activation of the auto-injector, such as up to 50 kN and more particularly in between 5N and 50 kN. FIGS. 1 and 2 show a medical device assembly having a glass cartridge protected by a protective sheath as further discussed herein. FIG. 3 shows experiments conducted on medical device assemblies having a protective sheath jacket and without the protective sheath jacket that have been either subjected to impacts and tumbling vibration conditions (i.e., conditioned configurations) or not subject to impacts/vibrations (i.e., unconditioned configurations). The experiments included 30 replicates per test condition. Generally, medical device assemblies having the glass cartridge with the disclosed sheath permit an increase in energy absorption from approximately 0.24 J (without the sheath jacket) to approximately 0.42 J (with the sheath jacket) under compressive forces, and a maximum compressive force increase from approximately 1.75 kN (without the sheath jacket) to approximately 2.5 kN (with the protective sheath jacket). For impact forces upon the assemblies, there is an increase from approximately 110 N mm (without the protective sheath jacket) to approximately 191N mm (with the protective sheath jacket) on the barrel and an increase from approximately 106N mm to approximately 129N mm.

FIG. 4 shows experiments conducted on the barrel, heel, and shoulder of the glass cartridge having a sheath with fluorinated ethylene propylene (FEP) material and an unsheathed glass cartridge (i.e., control). For each location (barrel, shoulder, heel), the sheath according to the disclosed subject matter outperformed the control for the impact force upon the medical device assembly as shown.

The protective sheath according to the disclosed subject matter protects the glass cartridge in different ways. The force absorption sheath can be positioned on the surface of the glass body such that the glass cartridge is protected from crushing forces applied to the barrel of the glass cartridge by the walls of the auto-injector. The force absorption sheath can be positioned on the surface of the glass body such that the glass cartridge is protected from abrasion during auto-injector activation and articulation of the glass cartridge in the auto-injector. The force absorption sheathing can be positioned on the surface of the glass cartridge such that it is protected from forces commonly occurring in demanding external environments. These environments can include police or other emergency kits, job sites, recreational and military environments or being worn on a person.

In demanding environmental use, the sheath protects the glass against shocks and impacts. As the sheathing also can cover the cartridge shoulder, it also serves as a shock absorbing element against the high energy impact experienced during the auto-injector activation process. During manufacturing, the sheathing inner diameter is recovered and is less than the initial diameter of the sheath pre-manufacture. The recovered sheath post-manufacture applies a hoop stress on the outer surface of the cartridge.

In one example, a three-foot drop simulation was conducted along the longitudinal axis of jacketed cartridges according to the disclosed subject matter in comparison with unjacketed cartridges. FIG. 5A depicts a finite element analysis of a defective unjacketed 1 mL cartridge, which experienced a first principal stress of 2.32e+07 at 3.695 microseconds after impact. In contrast, the jacketed cartridge according to the disclosed subject matter experienced a first principal stress of 2.125e+08 at the same interval as depicted by FIG. 5B.

In another example, a three-foot drop simulation was conducted along the transverse axis of jacketed cartridges according to the disclosed subject matter in comparison with unjacketed cartridges. FIG. 6A depicts a finite element analysis of a defective unjacketed 1 mL cartridge, which experienced a first principal stress of 2.125e+08 at 3.695 microseconds after impact. In contrast, the jacketed cartridge according to the disclosed subject matter experienced a first principal stress of 2.32e+07 at the same interval as depicted by FIG. 6B. This compressive force of the sheath upon the cartridge can induce additional propagation of otherwise hard to identify cracks. This aids in the quality control process and enables rejection of cartridges that might otherwise escape detection. A danger of permitting defective cartridges in the field is that such medical devices ultimately fail catastrophically at such cracks during forces generated during the autoinjector activation, which could lead to loss of life if the therapeutic or drug is not able to be delivered. In one embodiment, the sheathing can be optically transparent such that the contents of the glass cartridge remain visible at all times and optical inspection methods are viable. In other embodiments, the sheathing can be translucent or opaque to protect the cartridge contents from external effects such as ultraviolet rays.

Additional features and advantages of the embodiments of the glass containers described herein will be set forth in the detailed description which follows, and in part will be readily apparent to those skilled in the art from that description or recognized by practicing the embodiments described herein, including the detailed description which follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE FIGURES

Some embodiments of the disclosed subject matter are herein described, by example only, with reference to the accompanying drawings. With specific reference now to the drawings in detail, it is stressed that the particulars shown are by way of example and for purposes of illustrative discussion of embodiments of the disclosed subject matter. In this regard, the description taken with the drawings makes apparent to those skilled in the art how embodiments of the disclosed subject matter can be practiced.

FIG. 1 is a perspective phantom view of a force-shielding protective outer sheath affixed to a glass cartridge.

FIG. 2 is a cross-sectional view of a force-shielding protective outer sheath affixed to a glass cartridge.

FIG. 3 shows an interval plot of energy at yield of sample batch of assemblies according to the disclosed subject matter in comparison to cartridges without a sheath.

FIG. 4 is an interval plot of force upon an assembly at the barrel, heel, and shoulder locations of the sheath.

FIGS. 5A and 5B depict finite element analyses of a defective unjacketed and jacketed cartridge by way of a simulation with respect to the longitudinal axis of the device assembly, respectively.

FIGS. 6A and 6B depict finite element analyses of a defective unjacketed and jacketed cartridge by way of a simulation with respect to the transverse axis of the device assembly, respectively.

FIG. 7 is a cross-sectional view of an external object impacting the protective outer sheath and the protective outer sheath absorbing and dispersing the impact force.

FIG. 8 is a cross-sectional view of the protective sheath affixed to a glass cartridge embodied as an auto-injector subjected to a compressive force.

FIGS. 9A and 9B show a side partial phantom view and cross-sectional view about lines B-B of an embodiment where the sheath is applied to the glass cartridge in a manner to propagate hard to identify glass defects.

FIG. 9C shows an example of trauma imparted on a cartridge with a protective sheath according to the disclosed subject matter in comparison with a cartridge without a protective sheath.

FIG. 10 is a cross-sectional view of a shielded glass cartridge and needle-hub assembly. The shoulder of the glass cartridge is protected by a force absorbing sheath from impact of the needle-hub during auto-injector activation.

FIG. 11 depicts a histogram of burst pressure of a batch of assemblies according to the disclosed subject matter having a protective jacket in comparison to cartridges without a jacket.

FIG. 12 depicts an interval plot of burst pressure of a batch of assemblies according to the disclosed subject matter having a protective jacket in comparison to a cartridge without a jacket.

FIG. 13 is a cross-sectional view of a shielded glass cartridge and needle-hub assembly with arrows representing pressure applied on the glass cartridge during activation.

FIGS. 14 and 15 show examples of a cartridge failure with a jacket that has permanently deformed along the cartridge body and the cartridge shoulder, respectively.

FIG. 16 shows an example of a cartridge failure with a jacket that has permanently deformed along the cartridge body.

FIG. 17 depicts an assembly that has undergone stresses as evident by the marring on the jacket sheath and FIG. 18 is a magnified view of FIG. 17.

### DETAILED DESCRIPTION

Among those benefits and improvements that have been disclosed, other objects and advantages of the disclosed subject matter will become apparent from the following description taken in conjunction with the accompanying figures. Detailed embodiments of the presently disclosed subject matter are disclosed herein; however, it is to be understood that the disclosed embodiments are merely illustrative of the disclosed subject matter that can be embodied in various forms. In addition, each of the examples given in connection with the various embodiments of the disclosed subject matter which are intended to be illustrative, and not restrictive.

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though they can. Furthermore, the phrases “in another embodiment” and “in some other embodiments” as used herein do not necessarily refer to a different embodiment, although they can. Thus, as described below, various embodiments of the disclosed subject matter can be readily combined, without departing from the scope or spirit of the disclosed subject matter.

As used herein, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

Unless otherwise defined, all terms (including technical and scientific terms used herein have the same meaning as commonly understood by one having ordinary skill in the art to which this disclosed subject matter belongs. It will further be understood that terms, such as those defined, in commonly used dictionaries, should be interpreted as having a meaning that is consistent with their meaning in the context of the relevant art and the present disclosure and will not be interpreted in an idealized or overly formal sense unless expressly defined. The present disclosure describes an exemplary embodiment of a miniaturized wearable injection device.

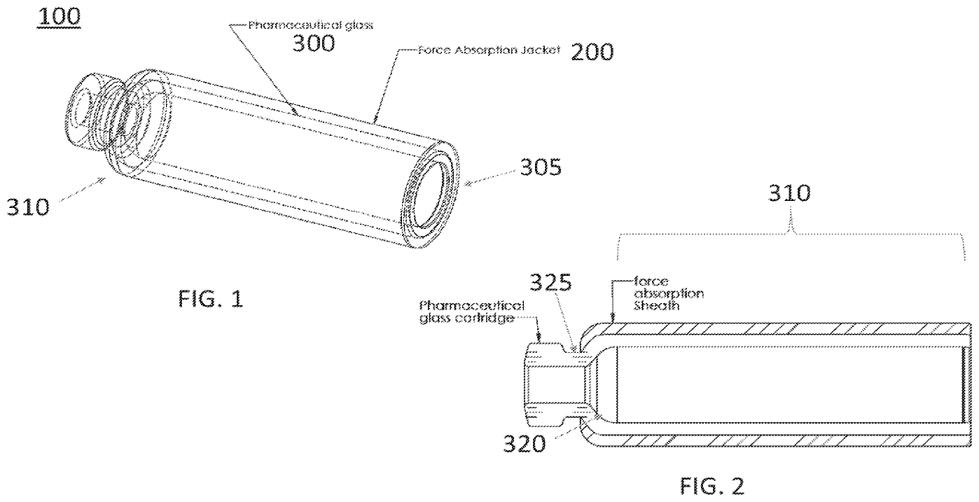

FIG. 1 shows a perspective view of an assembly 100 having the protective sheath 200 affixed to a glass cartridge 300. In this embodiment, the sheath is a monolithic unit but may be comprised of one or more subunits. The sheath can partially or entirely cover the exterior surface of the glass cartridge.

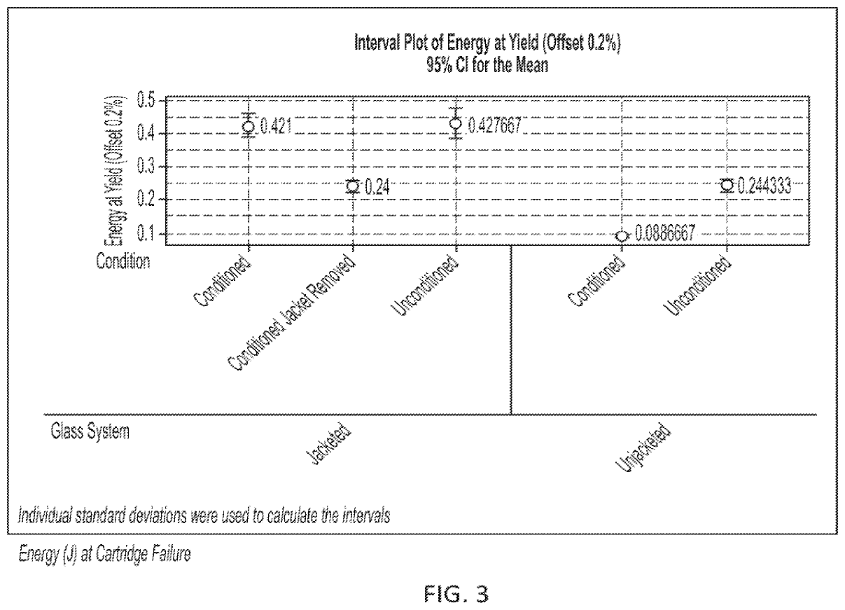

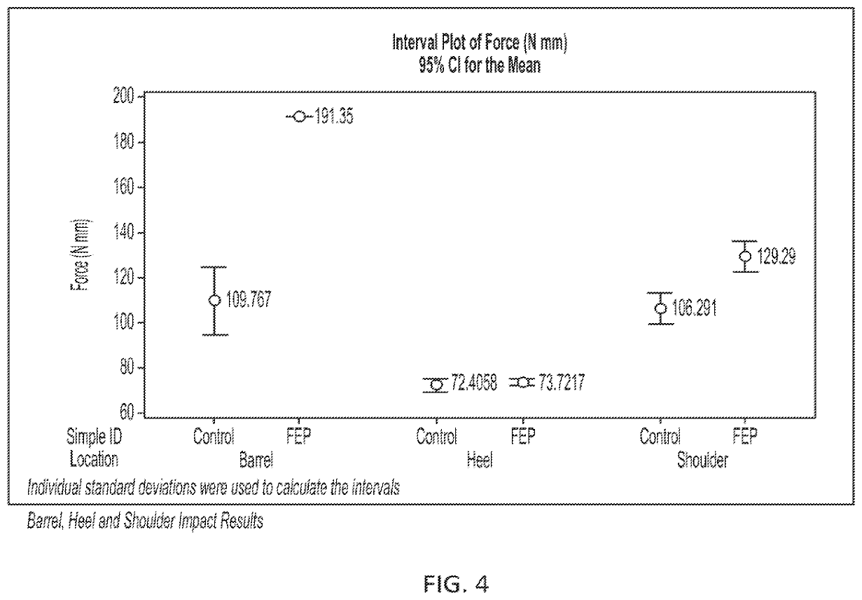

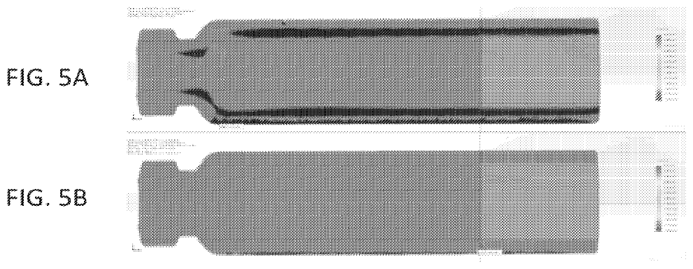

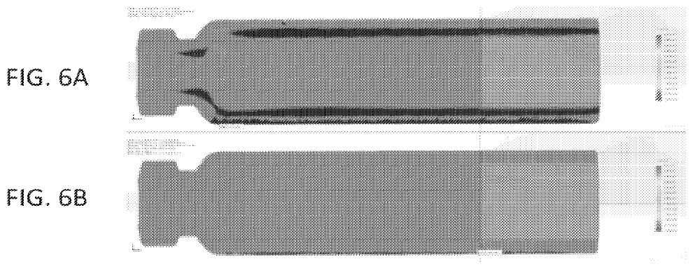

FIG. 2 is a side cross-sectional view of a protective sheath 200 coupled with a glass cartridge 300 that forms a force absorption outer protective sheath. As shown in the figure, the sheath extends from a first end 305 to a second end 310 of the body 315 of the cartridge. In this embodiment, the sheath further contacts the shoulder 320 of the cartridge approximate the neck 325 of the cartridge. Although one protective sheath is shown and described with respect to the figures for purposes of example, it is contemplated that a second (or more) sheath can be further coupled to the assembly.

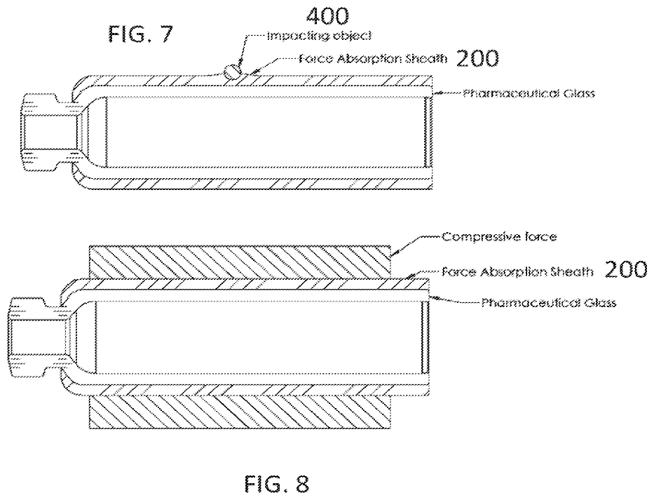

FIG. 7 is a cross-sectional view of an external object 400 impacting the protective outer sheath 200. As shown, the protective outer sheath absorbs and disperses the impact force. In such embodiment, the sheathed glass cartridge barrel has an increased impact strength due to the protective outer sheath in comparison with conventional coverings as discussed above.

In some embodiments, the protective sheath has a Shore hardness of greater than 70A. The hardness of the sheath allows for increase capability (such as but not limited to defect characterization and elimination of defective product) of visual quality process controls, which in turn creates greater reliability. Additionally, the hardness of the sheath allows the cartridge to maintain integrity in the event of a heel fracture during injector actuation and decreases the risk of a complete failure of drug delivery and a ‘no-dose’ event that could result in death.

The protective sheath can be transparent and difficult to perceive to the naked eye. The transparency and color of the protected cartridge can be assessed by measuring the light transmission of the sheathed container within a range of wavelengths between 400-700 nm using a spectrophotometer. Measurements are assessed by transmitting light into the cartridge normal to the wall such that the beam passes through the protective sheath twice, first when entering the container and then when exiting it. In some embodiments, the light transmission through the sheathed glass container can be greater than or equal to about 90% of a light transmission of an unsheathed glass container for wavelengths from about 400 nm to about 700 nm.

In some embodiments, the sheathed glass cartridge can be perceived as colorless and transparent to the naked human eye when viewed at any angle. Thus, the sheath permits inspection of the agent within the glass cartridge and permits visual inspection of the glass cartridge itself. In other embodiments, the protective sheath can be translucent or opaque to protect the cartridge contents from external effects such as ultraviolet rays.

In some embodiments, the sheathed glass cartridge has an increased compressive strength along the longitudinal shaft of the cartridge as best shown in FIG. 8.

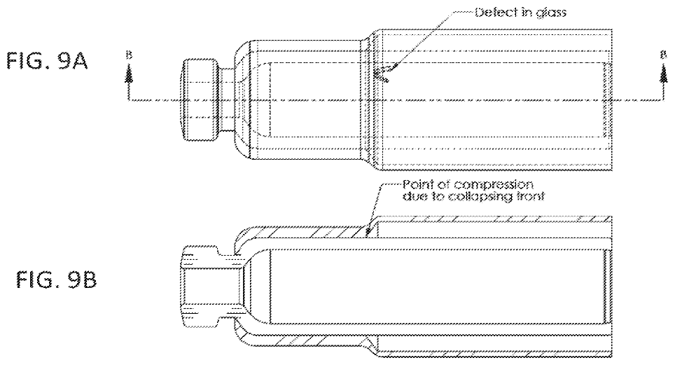

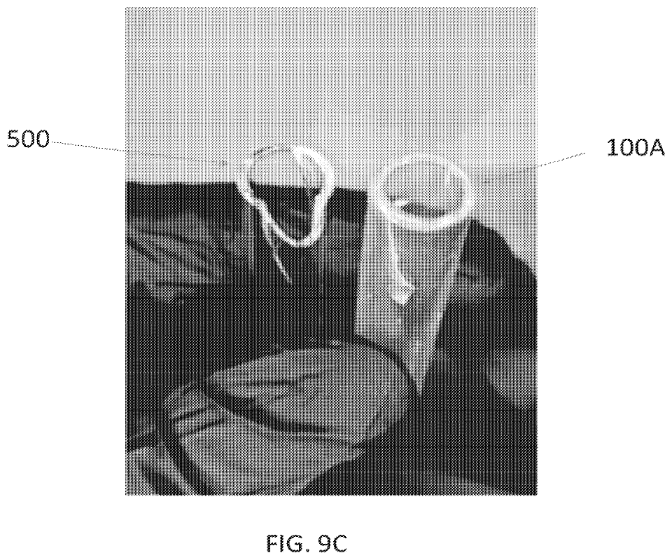

FIGS. 9A and 9B show a side view and cross-sectional view about lines B-B respectively. In these figures, the sheath is applied to the glass cartridge in a manner to propagate hard to identify glass defects such that the defective glass can be quickly eliminated in production by use of the protective sheath. In this embodiment of FIGS. 9A and 9B, the sheath is applied in a manner to propagate hard to optically identify defects that can be generated during the annealing process and eliminate these defective units in production. The compressive force applied during recovery or ‘shrinking’ of the outer sheath with respect to the cartridge can be sufficient to propagate or identify existing defects. As such, mechanical ‘inspection’ can be provided for every cartridge in a safe and cost-effective manner. Furthermore, the sheath can strengthen the structural integrity of a defective glass such that the therapeutic contained therein can still be delivered without the assembly resulting in complete failure (and possibly death of the patient). FIG. 9C depicts an example of an assembly 100A according to the disclosed subject matter having a protective sheath and a cartridge 500 that does not have a protective sheath. The same stresses were imparted on each device during testing, but the cartridge 500 that did not have a protective sheath resulted in complete failure, whereas the assembly 100A with the protective sheath could still deliver a therapeutic contained therein.

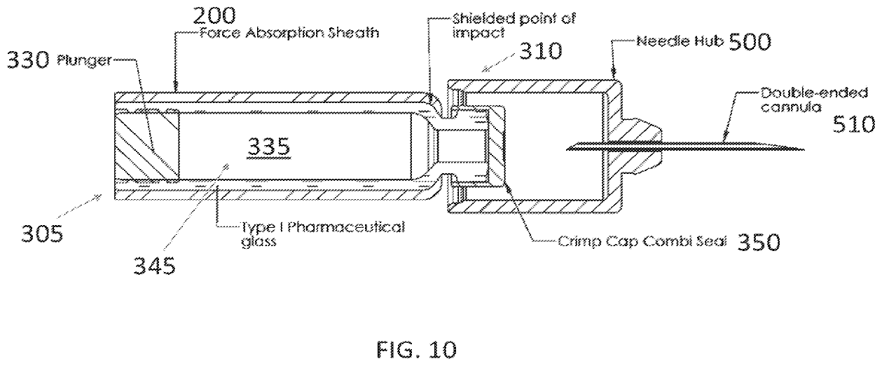

FIG. 10 depicts the glass cartridge with the protective sheath being coupled with a needle hub 500 having a double-ended cannula 510. In some embodiments, the shoulder of the sheathed glass cartridge has an increased impact strength, as represented by FIG. 10. The glass cartridge of this embodiment includes a Type I Pharmaceutical glass. The sheath circumscribes the cartridge along a longitudinal length thereof from a first end to a second end. The second end 310 of the sheath extends over the shoulders of the cartridge proximate the neck of the cartridge. Accordingly, the sheath-protected shoulders of the cartridge have a shielded point of impact such that a needle hub attached thereto does not add undue stress to the cartridge. As such, the needle hub interfaces with the protective sheath instead of the glass material of the glass cartridge. Thus, the sheathed glass cartridge shoulder has an increased impact strength.

As depicted in FIG. 10, a plunger 330 is provided within the glass cartridge to advance a pharmaceutical formulation or therapeutic 335 contained within the chamber 345 of the cartridge as desired. Accordingly, the agent or formulation 335 is disposed between the plunger and a dispensing end of the cartridge.

The dispensing end of the cartridge can include a crimp cap combi seal 350, although other seals are contemplated herein as known in the art. The dispensing end of the cartridge receives the needle hub 500 thereon, such as a needle hub having a double-ended cannula 510. The hub can be coupled to the dispensing end by any known technique, such as threaded connection or snap fit configuration. Once advanced onto the cartridge, the double-ended cannula can pierce the seal to dispense the formulation from the cartridge.

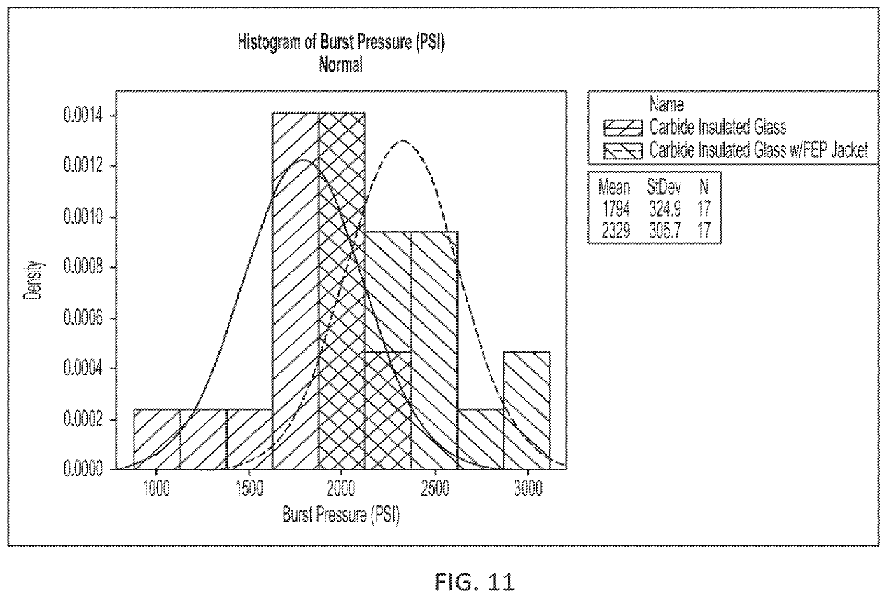

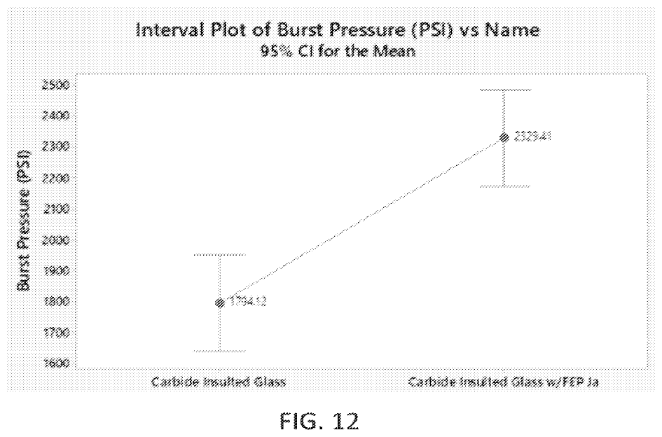

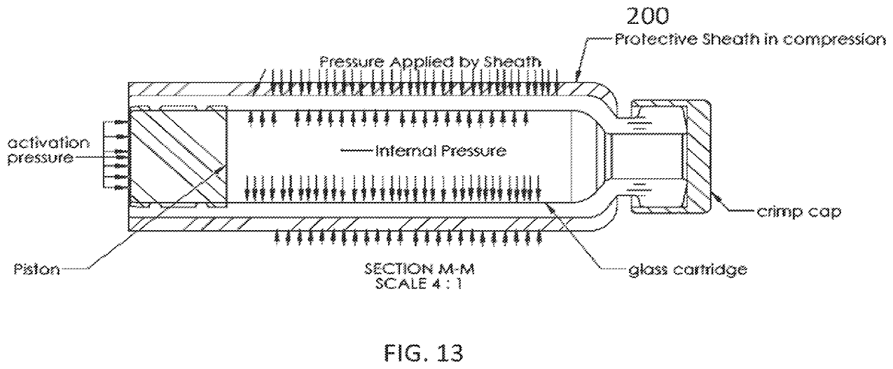

In some embodiments, the sheathed glass cartridge can withstand increased internal pressures before structural failure. By protecting the glass from insults, the surface of the glass cartridge is maintained/uncompromised and the resultant burst pressure is higher than a cartridge without a jacket, as depicted by data of FIG. 11 and FIG. 12. The experiments included 17 replicates per test condition. The plunger can be advanced from the first end of the cartridge towards the second end of the cartridge, as desired by a user or as an unintended consequence of external environmental factors. Pressures can be generated by the release of a stored energy component, include but are not limited to a spring, compressed gas, or chemical decomposition. As shown in FIG. 13, the plunger exerts activation pressure and force from the first end towards the second end of the cartridge. As such, the plunger acts as a piston. The added pressure from the plunger can increase the internal pressure in the chamber of the cartridge, such that first principal stress can be exerted on an inner surface of the cartridge. To counterbalance such internal pressure, the protective sheath can apply compressive forces to an external surface of the cartridge, as shown in FIG. 13.

The sheathed glass cartridge can withstand increased external pressures before structural failure as shown in FIG. 13. The sheath does not generate any internal pressure upon the glass cartridge. In one embodiment, the sheath begins to separate from the cartridge at 150 psi. The pressures required to cause a failure in the cartridge can range based on the volume of the cartridge, thickness of the sheath, and thickness of the cartridge wall thickness, such as up to 2000 psi for a 1 ml cartridge. Pressures can be generated by an external environmental condition.

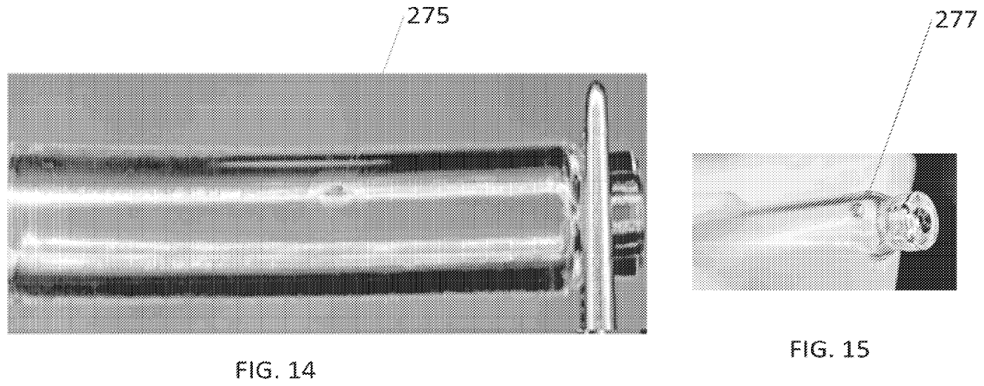

In some embodiments, the protective sheath is not permanently bonded directly to the glass cartridge. Rather, the sheath is held in place by the mechanical compression applied by the recovered sheath on the cartridge exterior. By being unbonded, the sheath has superior impact resistance by allowing micromovement or small amounts of slippage of the sheath over the cartridge. The unbonded and rigid nature of the sheath allows for better impact resistance via redirecting some energy into a glancing impact. Additionally, the micromovements paired with the rigidity characteristic provide characteristic marring that identifies a history of abuse, as described in FIGS. 14-18.

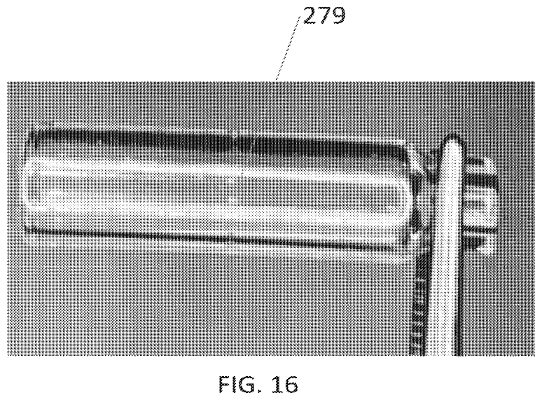

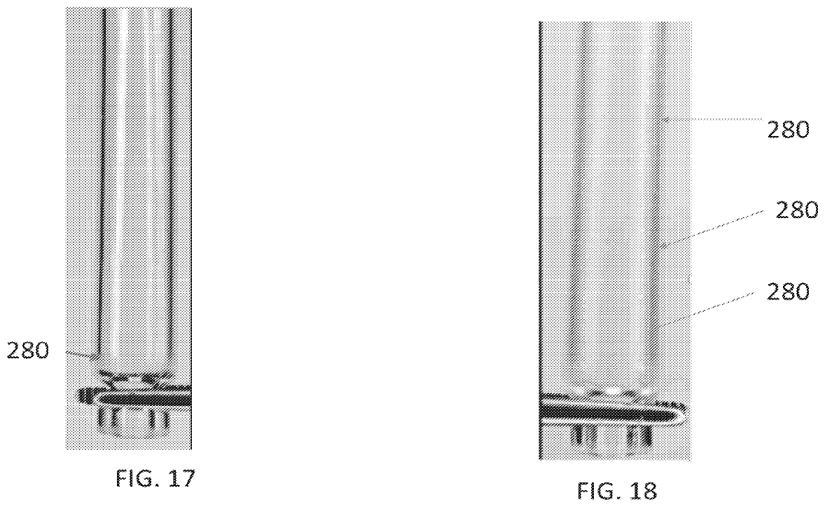

As disclosed herein, the protective sheath can comprise a number of suitable materials. Heat and/or cold shrink polymers can be used for the compressive sheath that are recovered over the cartridge post-manufacture. Such sheaths are uncoupled such that the sheaths are not directly and permanently bonded to the cartridge in a static relationship between the sheath and the cartridge. Rather, the dynamic relationship between the sheath and the cartridge permits slippage and micromovement as discussed herein. Although the disclosed sheath is a rigid unit, the sheath can exhibit permanent deformation at glass cartridge failures (such as at certain crack locations) and an enclosed “bubble” can form in the sheath at such location. FIG. 14 provides an example of a sheath having a bubble 275 at a crack location along the barrel of the cartridge and FIG. 15 provides an example of a sheath having a bubble 277 proximate a shoulder of the cartridge. As the sheath cannot elastically deform (unlike silicone conventional coverings), the sheath is configured to permanently deform to enable an otherwise defective medical device to administer therapeutic agent therefrom. FIG. 16 shows an example of a cartridge failure with a jacket that has permanently deformed at a location 279 along the cartridge body. The assembly of FIG. 17 along with a magnified view of the assembly of FIG. 18 depict cartridge damage from vibration at various locations 280 as shown. The protective sheath is marred and shows a visual indicator of the impacts showing a clear history of rough handling. Additionally, with the dynamic configuration, there remains flexibility of when the sheath is applied to the cartridge during the manufacturing process and advantages to further protecting drug loading or assembly of the crimp-cap/plunger components given the flexibility of when the sheath can be applied to the cartridge.

In some embodiments, the glass is protected by a fluoropolymer sheath that does not further require a coupling agent. In another embodiment, the polymer chemical composition of the sheath may comprise a fluoropolymer. The fluoropolymer may be a copolymer wherein both monomers are highly fluorinated. In one embodiment, the polymer chemical composition comprises an amorphous fluoropolymer, such as, but not limited to, Teflon. In another embodiment, the polymer chemical composition can comprise perfluoroalkoxy (PFA) resin particles, but not limited thereto. In another embodiment, the polymer chemical composition can comprise fluorinated ethylene propylene (FEP) resin particles.

In some embodiments, the sheath comprises fluoroelastomers or fluorocarbons (FKM), such as but not limited to Viton, and are highly fluorinated polymers that are suitable for continuous use at elevated temperatures. Various grades are available for the sheath, including copolymer and terpolymers and also tetrafluoroethylene, hexafluoropropylene, and vinylidene fluoride. Such sheaths of such composition have limited resistance to steam, hot water, and polar fluids such as strong organic acids (e.g. formic acid), methanol, ketones, ammonia and some amines.

In some embodiments, the sheath comprises chloroprene such as chloroprene available in commercial grades that are mostly trans-1,4-polychloroprene produced by free-radical emulsion polymerization of 2-chloro-1,3-butadiene. The chlorine in the polymer reduces the reactivity to many oxidizing agents and thus improves its chemical resistance. Due to its low reactivity, sheaths of such composition display suitable resistance to ozone cracking, heat aging and chemical attack. For example, sheaths of such composition have good resistance to many chlorofluorocarbons, aliphatic hydrocarbons, mineral oils, greases and ozone and substantial flame resistance. In fact, chloroprene is one of the few rubbers that are self-extinguishing.

In some embodiments, the sheath comprises thermoplastic vulcanizates also known as the polymer chemical Santoprene®, a vulcanized rubber with the stable physical properties of a thermoplastic. The sheaths comprising Santoprene TPV grades cab range from a hardness of 35 Shore A up to 50 Shore D. In some embodiments, the sheaths comprising Santoprene (TPV) can be processed using conventional thermoplastic processes such as injection molding, blow molding and extrusion.

Table 1 provides examples of suitable materials of construction for the sheath according to the disclosed subject matter along with their property characteristics.

TABLE 1

| | FEP | PFA | PVC | Polyolefin | PTFE |
|---|---|---|---|---|---|
| Initial ID (mm) | 9.53 | 9.53 | 9.53 | 9.53 | 9.53 |
| Recovered ID (mm) | 8.65 | 8.65 | 8.65 | 8.65 | 8.65 |
| Initial OD (mm) | 9.8 | 9.8 | 10 | 9.8 | 9.8 |
| Recovered OD (mm) | 9.35 | 9.35 | 9.35 | 9.42 | 8.8 |
| Shrink Ratio | 1.6:1 | 1.6:1 | 2 to 1 | 2 to 1 | 4 to 1 |
| Wall Thickness (mm) | 0.3 | 0.3 | 0.35 | 0.3 | 0.05 |
| Minimum Heat Shrink | 215 | 110 | 100 | 100 | 340 |
| Temp ° C. | | | | | |
| Maximum Heat Shrink | 288 | 260 | 240 | 150 | 260 |
| working Temp ° C. | | | | | |
| tensile strength (psi) | 3050 | 4500 | 3000 | 3770 | 4350 |
| Brittleness temp ° C. | −260 | −260 | −28 | −90 | −200 |
| Sterilization method | ETO & | ETO & | ETO & | ETO | ETO & |
| | Steam | Steam | Steam | | Steam |

The protective sheath can include materials that have sufficient rigidity, such as those having a hardness/durometer within the semi-rigid to rigid plastics family of SHORE D 30-60. In one embodiment, the sheath comprises a Fluorinated Ethylene Propylene (FEP) polymer that can furthermore include material to fill the FEP extrusions to prevent UV penetration, thereby further protecting the contents of the glass cartridge. For purposes of examples, such fillers can include radio-opaque (bismuth trioxide and bismuth oxychloride), carbon, pigments and the like and the FEP can be provided by Zeus Inc. Suitable materials for the disclosed sheath can have a high lubricity (PTFE)/low Coefficient of Friction (CoF) of <0.06, which allows for rapid movement of product through high-speed manufacturing lines. By way of contrast, cartridges with conventional silicone coverings have a CoF of approximately 1 and exhibit degrees of flexibility not desirable for a protective sheath as embodied herein. Suitable materials for the disclosed sheath can have a low water absorption percentage. In one embodiment, the percent of water absorption for an FEP sheath has a value of <0.01%, which can assist with stability and extend service-life. By way of contrast, conventional silicone coverings have a water absorption value of approximately 1%).

The method of manufacturing the medical device assembly has been described throughout in conjunction with the assembly and protective sheath. As noted, a method of manufacturing a medical device assembly is described including providing a glass cartridge to house a substance therein and coupling a detachable and unbonded sheath to the glass cartridge along a longitudinal length thereof by at least one of heat or cold shrinking. The tolerances or the protective sheath coupled to the cartridge are sufficiently narrow, for example to sufficiently cover the heel of the cartridge after shrinkage. Because the sheath collapses during a shearing cut (such as by scissors or the like) and shrinks during a wire/laser cut, the tubing can be cut with a mandril supporting the interior of the sheath. In one embodiment, the sheath diameter can be recovered and cured from the heel of the cartridge first and progresses forward to the shoulder for positioning of the sheath on the cartridge.

While the disclosed subject matter is described herein in terms of certain examples and embodiments, those skilled in the art will recognize that various modifications and improvements can be made to the disclosed subject matter without departing from the scope thereof. Moreover, although individual features of one embodiment of the disclosed subject matter can be discussed herein or shown in the drawings of the one embodiment and not in other embodiments, it should be apparent that individual features of one embodiment can be combined with one or more features of another embodiment or features from a plurality of embodiments.

In addition to the various embodiments depicted and claimed, the disclosed subject matter is also directed to other embodiments having any other possible combination of the features disclosed and claimed herein. As such, the particular features presented herein can be combined with each other in other manners within the scope of the disclosed subject matter such that the disclosed subject matter includes any suitable combination of the features disclosed herein. Furthermore, although reference is made to sheath for a glass cartridge throughout this disclosure, other suitable devices likewise can be protected using the compressive sheath disclosed herein, such as but not limited to prefilled syringes. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the device, method and system of the disclosed subject matter without departing from the spirit or scope of the disclosed subject matter. Thus, it is intended that the disclosed subject matter include modifications and variations that are within the scope of the appended claims and their equivalents.

1. A medical device assembly, comprising:
a glass cartridge to house a substance therein; and
an unbonded protective sheath detachably coupled to the glass cartridge and movable by micromovements with respect to the glass cartridge, wherein the sheath imparts a compressive force and hoop stress upon the glass cartridge and is configured to at least one of protect the glass cartridge, increase visual detection of trauma to the medical device assembly, and increase visual detection of defects of the glass cartridge.

2. The medical device assembly of claim 1, wherein the sheath has a thickness dimension of less than or equal to 0.2 mm.

3. The medical device assembly of claim 1, wherein the sheath is coupled to an outer surface of the body of the glass cartridge.

4. The medical device assembly of claim 1, wherein the sheath has an inner surface and an outer surface, wherein the sheath is rigid and configured to permanently deform when a force is exerted upon the inner surface of the sheath.

5. The medical device assembly of claim 4, wherein the sheath permanently deforms at a location of the force exerted upon the inner surface of the sheath to create a bubble, wherein the sheath contains any substance that exits the glass cartridge therein.

6. The medical device assembly of claim 1, wherein sheath is configured to support the glass cartridge and configured to prevent failure of administering the substance from the glass cartridge.

7. The medical device assembly of claim 1, wherein the compressive force can range from approximately 75 psi to approximately 150 psi.

8. The medical device assembly of claim 1, wherein the sheath is coupled to the glass cartridge by the compressive force alone.

9. The medical device assembly of claim 1, wherein the sheath is configured to absorb an external impact force ranging up to 50 kN without the glass cartridge breaking and the sheath is configured to disperse the impact force along a body of the glass cartridge.

10. The medical device assembly of claim 1, wherein the sheath is a monolithic unit.

11. The medical device assembly of claim 1, wherein the sheath has a Shore hardness of greater than 70A.

12. The medical device assembly of claim 1, wherein the glass cartridge includes a longitudinal body having a first end and a second end, a shoulder section and a neck, wherein the sheath is disposed over at least the longitudinal body from the first end to the second end to protect the glass cartridge.

13. The medical device assembly of claim 12, wherein the sheath is disposed over the shoulder section and the neck of the glass cartridge.

14. The medical device assembly of claim 1, wherein the sheath propagates cracks of the glass cartridge for quality control inspection.

15. The medical device assembly of claim 1, wherein the substance is a therapeutic agent and the medical device is an auto-injector.

16. The medical device assembly of claim 1, further comprising a plunger to cooperate with the glass cartridge to advance agent within a chamber of the cartridge, wherein the plunger is configured to exert an activation pressure and force within the cartridge and the sheath is configured to apply compressive force to the cartridge to counterbalance the activation pressure and force upon the glass cartridge.

17. The medical device assembly of claim 1, wherein the sheath comprises a transparent material to permit inspection of the agent within the glass cartridge.

18. The medical device assembly of claim 1, wherein the sheath comprises at least one of a fluoropolymer, chloroprene, thermoplastic vulcanizate, fluoroelastomer, and fluorocarbon.

19. A protective sheath for a glass cartridge, comprising
a rigid material including at least one of a fluoropolymer, chloroprene, thermoplastic vulcanizate, fluoroelastomer, and fluorocarbon, wherein the sheath is configured to be detachably coupled with and unbonded to a glass cartridge having a therapeutic agent therein, wherein the protective sheath is configured to move by micromovements with respect to the cartridge and the sheath is configured to impart a compressive force and hoop stress upon the glass cartridge and to at least one of protect the glass cartridge, increase visual detection of trauma to the medical device assembly, and increase visual detection of defects of the glass cartridge.

20. A method of manufacturing a medical device assembly, comprising:
providing a glass cartridge to house a substance therein;
coupling a detachable and unbonded sheath to the glass cartridge along a longitudinal length thereof by at least one of heat shrinking or cold shrinking, wherein the sheath is movable by micromovements with respect to the cartridge and imparts a compressive force and hoop stress upon the glass cartridge, wherein the sheath is configured to at least one of protect the glass cartridge, increase visual detection of trauma to the medical device assembly, and increase visual detection of defects of the glass cartridge.
